(19) United States
(12) Patent Application Publication
(10) Pub. No.: US 2016/0376764 A1
(43) Pub. Date: Dec. 29, 2016

(54) METHOD FOR WATERPROOFING BELOW-GRADE STRUCTURES

(71) Applicant: THUR S.R.L., Verona (IT)

(72) Inventors: Mariapia PASTOR, Bosco Chiesanuova (IT); Andrea BIRTELE, Verona (IT)

(21) Appl. No.: 15/039,783

(22) PCT Filed: Nov. 13, 2014
(86) PCT No.: PCT/EP2014/074454

§ 371 Date: May 26, 2016

(30) Foreign Application Priority Data
Nov. 27, 2013 (IT) VR2013A000258

(51) Int. Cl. E02D 31/02; E02D 31/04; E02D 31/10; B29C 44/12; E02D 19/00; G01N 33/24; G01N 27/12; B29C 45/14; E02D 31/06; E02D 3/12

(52) U.S. Cl. CPC E02D 31/02; E02D 31/06; E02D 31/04; E02D 31/10; E02D 3/12; E02D 19/00; G01N 33/24; G01N 27/121; B29C 45/14; B29C 44/12; G01M 3/40

(57) ABSTRACT

A method for waterproofing below-grade structures includes at least one step of injecting at least one compound adjacent to the surface that is directed toward the outside of a region of soil arranged adjacent to a below-grade structure to be waterproofed. The method further includes at least one step of measuring the electrical resistivity of the at least one region of soil subjected to the injection step. The at least one measurement step is adapted to detect the portions of the region of soil that are affected by the injection of the at least one compound.

### CROSS-REFERENCE TO RELATED APPLICATIONS

This application is related to and claims the benefit of Italian Patent Application No. VR2013A000258, filed on Nov. 27, 2013 and PCT Application No. PCT/EP2014/074454, filed on Nov. 13, 2014, the contents of which are herein incorporated by reference in their entirety.

### TECHNICAL FIELD

The present disclosure relates to a method for waterproofing below-grade structures such as masonry against earth or flooring.

### BACKGROUND

Techniques of (retrospective) waterproofing with injections of compounds, such as silicate mixtures, have already been used for many years. They can be carried out both inside the masonry facing that is to be waterproofed, in order to saturate the empty spaces that are present, and at the rear of the masonry, for the purpose of creating a waterproofing barrier between masonry and soil.

The products used can be of several different types, for example: polyurethane resins, acrylic resins or silicate mixtures.

Usually, injections of resins and/or gels which are executed behind masonry for waterproofing purposes are carried out according to the following operational steps:
drilling of the masonry according to a regular pattern;
positioning of tubes for injection in the holes;
carrying out of the injections starting from the areas arranged at lower vertical levels and proceeding by successive horizontal alignments toward the top of the masonry facing.

The substances injected, initially in the liquid state, solidify or gel more or less rapidly, thus becoming waterproof.

The times necessary for the injected substance to pass from the liquid state to the solid (or gel) state can vary, as a function of the type of reagent used, from a few seconds to a few hours.

The testing methods used in order to verify the success of the operation, i.e. in order to verify that the waterproofing barrier has a spatial continuity that follows the progression of the surface of the treated masonry, are generally of the indirect type and are empirical in character, and consequently they are often influenced by the practice and experience of the technician.

For the purposes of example, in the event of heavy incursions of water, one is limited to visually checking the effective interruption of the water flow through the masonry facing in order to validate the success of the operation.

If incursions of water are not present or if it is desired to provide a continuous waterproof barrier in order to block, for example, future rises in the aquifer, one is limited to using some contrivances during the step of injecting the product.

According to a fairly consolidated practice, an injection sequence is carried out that involves proceeding, for vertical masonry, by successive rows, starting at the bottom and proceeding upwardly, and stopping the insertion of the material when the injected product flows out of the lateral holes, and in particular from those positioned at greater heights.

Even with the use of such contrivances in the installation of the product, it cannot be ruled out that some areas of masonry will not be reached by the injected product, which may arrange itself only at the areas of greatest permeability.

Such problems are present to a greater extent for treatments carried out below horizontal surfaces.

### SUMMARY

The aim of the present disclosure is to solve the above mentioned problems and overcome the drawbacks, by providing a method for waterproofing below-grade structures, such as masonry against earth or flooring, which makes it possible to effectively waterproof a below-grade structure while verifying, at the same time, the effective continuity of the waterproofing barrier.

Within this aim, the disclosure provides a method for waterproofing below-grade structures such as masonry against earth or flooring which makes it possible to intervene in order to supplement the operation in a targeted manner even at a later stage if it is found that some areas are missing the waterproofing barrier.

### BRIEF DESCRIPTION OF THE DRAWINGS

Further characteristics and advantages of the disclosure will become better apparent from the description of some preferred, but not exclusive, embodiments of the method for waterproofmg below-grade structures such as masonry against earth or flooring according to the disclosure, which are illustrated by way of non-limiting example in the accompanying drawings wherein:

FIG. 1 is a view from the outside of a below-grade structure to be waterproofed;

FIG. 2 is a cross-sectional view of the masonry structure taken along the line II-II in FIG. 1;

FIG. 3 is a view from the inside of the masonry in FIG. 1 into which electrodes have been inserted, through the masonry, for detecting the resistivity of the earth adjacent to of the masonry;

FIG. 4 is a cross-sectional view of the below-grade structure taken along the line IV-IV in FIG. 3;

FIG. 5 is a view similar to that of FIG. 3 in which first injection tubes have been inserted;

FIG. 6 is a cross-sectional view of the below-grade structure taken along the line VI-VI in FIG. 5;

FIG. 7 is a view similar to that of FIG. 1 in which a first compound has been injected through the first injection tubes;

FIG. 8 is a cross-sectional view of the below-grade structure taken along the line VIII-VIII in FIG. 7;

FIG. 9 is a view similar to that of FIG. 7 in which the second injection tubes have been inserted through the below-grade structure, and in which a second compound has been injected through the second tubes; and

FIG. 10 is a cross-sectional view of the below-grade structure taken along the line X-X in FIG. 9.

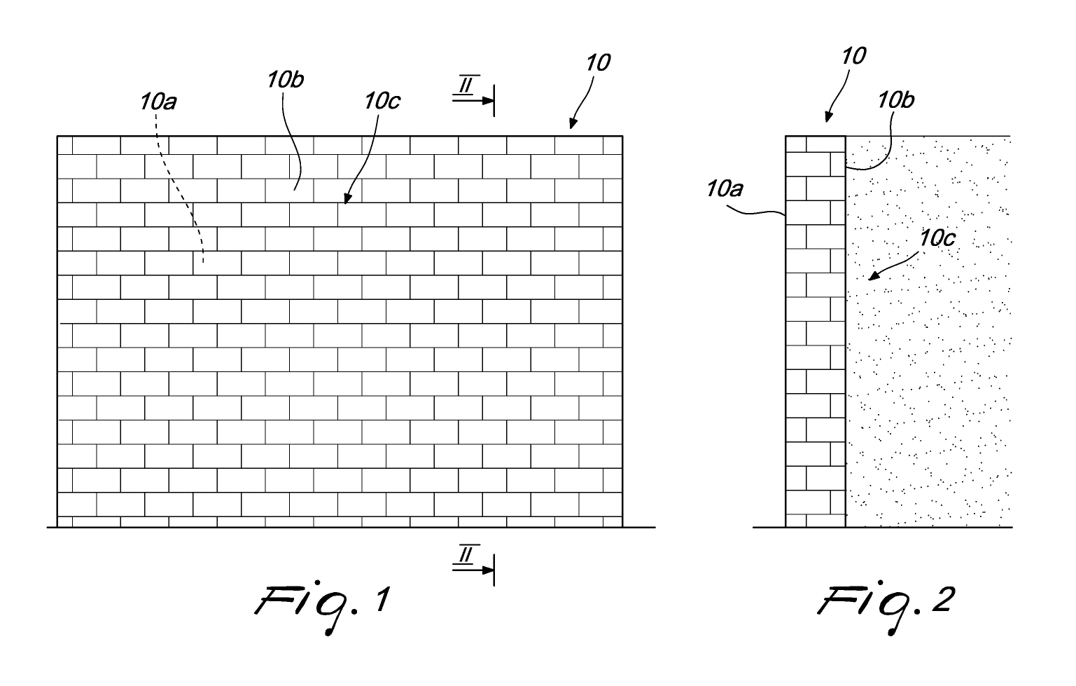

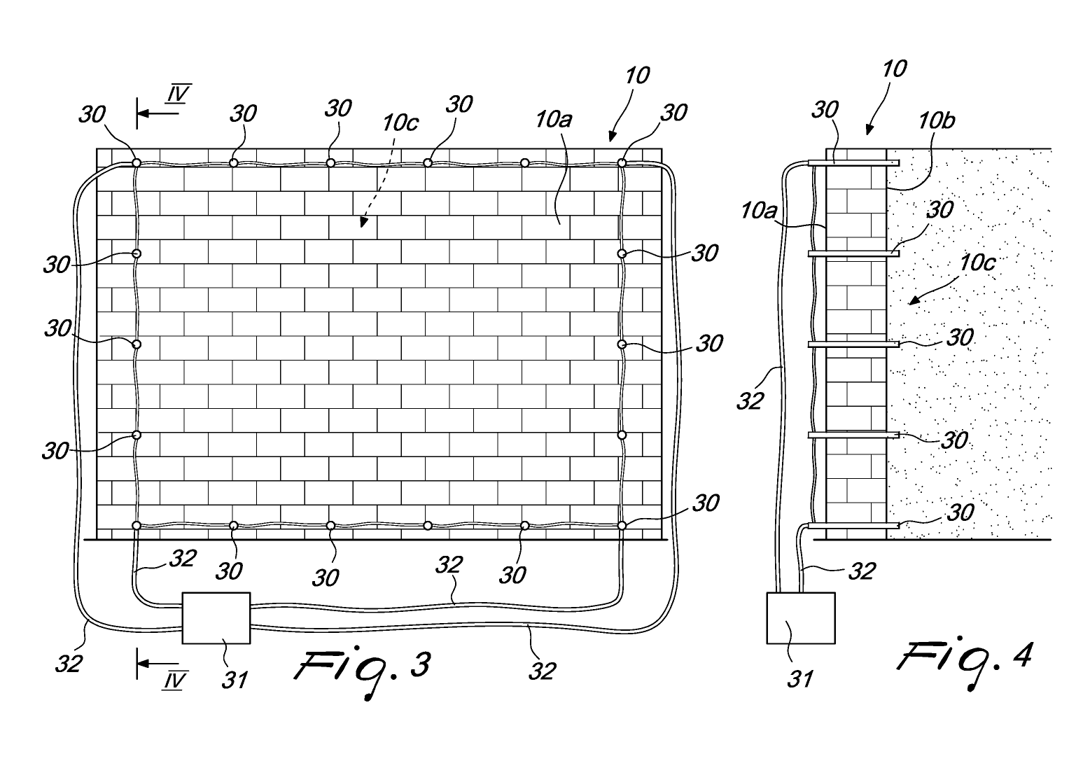

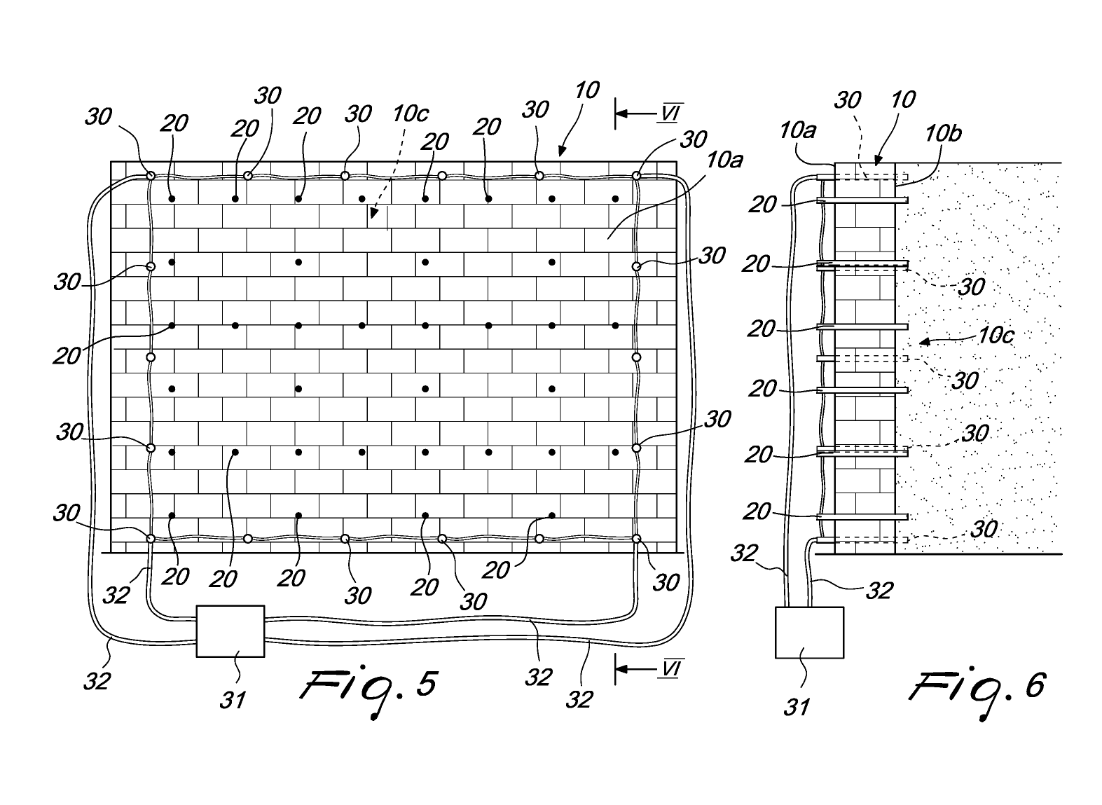

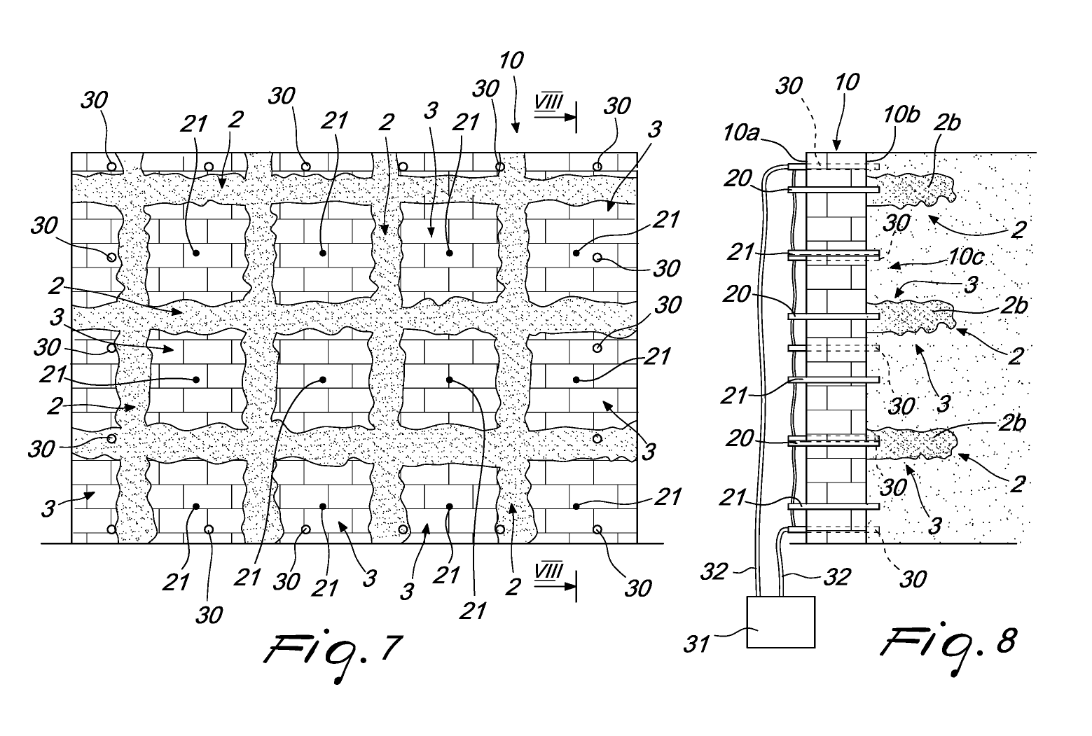

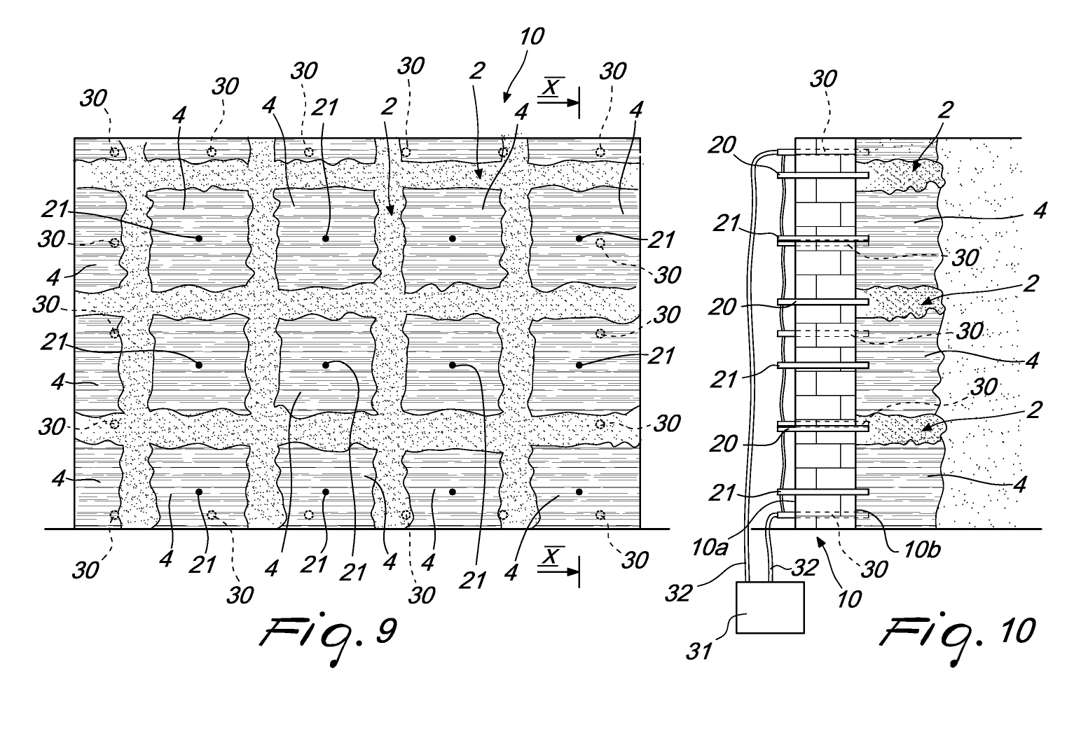

In the embodiments illustrated, individual characteristics shown in relation to specific examples may in reality be interchanged with other, different characteristics, existing in other embodiments.

### DETAILED DESCRIPTION OF THE DRAWINGS

With reference to the figures, the present disclosure relates to a method for waterproofing below-grade structures 10 such as, for example, masonry against earth or flooring.

The method includes the following steps:
at least one step of injecting at least one compound 2, 4 adjacent to the surface 10b that is directed toward the outside of a region of soil 10c arranged adjacent to a below-grade structure 10 to be waterproofed;
at least one step of measuring the electrical resistivity of the region of soil 10c subjected to the injection step.

In particular, the measurement step is adapted to detect the portions of the region of soil which are affected by the injection of the compound or compounds 2, 4.

Advantageously, the compound and/or the compounds 2, 4 injected in order to obtain the waterproofing of the below-grade structure 10 has a higher resistivity than water.

Preferably, such compound(s) 2, 4 has/have a resistivity that is at least ten times greater than the resistivity of water.

According to a preferred embodiment, the method involves a first step of measuring the electrical resistivity of the region of soil to be waterproofed before performing the injection step and at least a second step of measuring the electrical resistivity of the same region of soil 10c after carrying out the injection step.

Specifically, the method includes: a step of comparison between the values of the electrical resistivity of respective portions of the region of soil 10c to be waterproofed, which are measured during the first measurement step and during the second measurement step in order to detect whether each portion has been affected by the injection of compound(s) 2, 4.

With reference to the embodiment shown in the figures, the compound 2, 4 comprises an expanding compound 2 and/or a diffusing compound 4.

The expanding compound 2 can be selected from the group comprising:
a polyurethane resin;
a urea resin;
a silicone foam;

or mixtures thereof, while the diffusing compound 4 can be selected from the group comprising:
a silicate mixture;
a polyester resin;
an epoxy resin;

or mixtures thereof.

It is possible for the injection step to be executed at first portions of the region of soil to be waterproofed which have a lower resistivity than a first reference value.

Such first portions can, for example, be identified as a consequence of the measurement step and, specifically, of the first measurement step.

Especially if diffusing or permeating compounds are used, it is advisable that these compounds have components and/or additives that are adapted to increase the resistivity of the compound.

Such components and/or additives can be adapted to increase the resistivity of the compound when such compound solidifies or gels.

For the purposes of example, such components and/or additives can be selected from the group comprising:
acrylates;
acrylic copolymers;

or mixtures thereof.

Conveniently, the step of measuring the electrical resistivity of the region of soil that has been subjected to the injection step comprises a step of insertion, through respective openings for passing through the masonry and along the perimeter of the region of soil to be waterproofed, of a plurality of detection electrodes 30 that are connected, for example by way of connecting cables 32, to a georesistivity meter 31.

Advantageously, the detection electrodes 30 have a covering made of insulating material at least at the portion that is designed, during use, to arrange itself along the transverse extension of the masonry so as to prevent reading errors owing to points of contact between the outer surface of the detection electrode 30 and the masonry.

In particular, the georesistivity meter 31 comprises a device for administering current into the ground connected, for example, to a battery or to a current generator.

The georesistivity meter 31 is likewise connected to the plurality of detection electrodes 30 and, in particular, to at least two current electrodes and to at least two power electrodes.

The georesistivity meter further has an apparatus for measuring the intensity of the current input into the ground by way of the current electrodes and of the potential difference between the power electrodes.

Advantageously, the step of measuring the electrical resistivity of the region of soil that has been subjected to the injection step uses a plurality of detection electrodes 30, for example sixteen, which are distributed along a profile at a close distance (generally a few dozen centimeters).

By way of an adapted device, based on one or more multi-electrode cables and corresponding switching or exchange devices, the detection electrodes 30 are connected to the data acquisition unit/energizer of the georesistivity meter 31 so as to be able to function alternatively as current electrodes or as potential electrodes.

In this manner the measurements along a profile can proceed automatically according to the desired sequence, thus providing the resistivity values, including at different depths and locations along such profile.

Inter alia, since the method provides a way to measure the resistivity of the portion of ground immediately adjacent to the masonry, it has been found that it is possible to obtain, while limiting the measuring of the resistivity to a thickness of earth of a few dozen centimeters, extremely precise values that make it possible to identify, if present, the portions of earth that are not affected by the injections.

With reference to the figures, the method can involve:
a step of injecting an expanding compound 2 adjacent to the surface 10b directed toward the outside of a masonry structure 10 to be waterproofed so as to define at least one portion of confinement;
a step of injecting a diffusing, or permeating compound 4, which is designed to solidify or gel adjacent to the surface 10b directed toward the outside of the masonry structure 10 and at a completion region 3 that is delimited by the confinement portions.

Conveniently, the method can involve, in sequence, the step of injecting the expanding compound 2 and the step of injecting the diffusing or permeating compound 4.

Delving deeper into the details, the method comprises: a step of insertion of first injection tubes 20 of the expanding compound 2 and a step of insertion of second injection tubes 21 of the diffusing compound 4.

The first injection tubes 20 and the second injection tubes 21 can be inserted from inside the building, by drilling the below-grade structure 10 from the surface directed toward the inside 10a, in order to emerge, with the dispensing tip thereof, adjacent to the surface 10b directed toward the outside of the below-grade structure 10 to be waterproofed.

In this case, the first injection tubes 20 and the second injection tubes 21 extend along a longitudinal direction that is substantially perpendicular to the plane of arrangement of the below-grade structure 10 to be waterproofed.

Alternatively, the first injection tubes 20 and the second injection tubes 21 can be inserted from the outside of the building in order to emerge adjacent to the surface 10b directed toward the outside of the below-grade structure 10 to be waterproofed.

In this case, the first and second injection tubes extend along a longitudinal direction that is substantially parallel to the plane of arrangement of the below-grade structure 10 to be waterproofed.

Once the injection of the compound or compounds has been completed, the method involves the removal of the first and second injection tubes 20, 21.

The first tubes 20 can likewise be left in place because their use is often rendered impossible by the fact that the expanding compound also solidifies inside them.

The method according to the disclosure can involve, subsequently to the injection step and to the measurement step, a further injection step at the portions where the resistivity measured is lower than the first reference value or than a second reference value.

In this case, in fact, it is probable that during the preceding injection step or steps, the compound has not reached such portions.

In practice the materials employed may be any, according to requirements.

Moreover, all the details may be substituted by other, technically equivalent elements.

1-12. (canceled)

13. A method for waterproofing below-grade structures, the method including the following steps:
at least one step of injecting at least one compound adjacent to a surface that is directed toward the outside of a region of soil arranged adjacent to a below-grade structure to be waterproofed, and
at least one step of measuring an electrical resistivity of said at least one region of soil subjected to said injection step, said at least one measurement step being adapted to detect portions of said region of soil that are affected by the injection of said at least one compound.

14. The method for waterproofing below-grade structures according to claim 13, wherein said compound has a higher resistivity than water.

15. The method for waterproofing below-grade structures according to claim 13, wherein said at least one step of measuring the electrical resistivity includes a first step of measuring the electrical resistivity of said at least one region of soil before said at least one injection step and at least one second step of measuring said electrical resistivity of said region of soil after said at least one injection step, the method further including a step of comparison between values of the electrical resistivity of respective portions of said region of soil which are measured during said first measurement step and during said second measurement step in order to detect whether each one of said portions has been affected by the injection of said compound.

16. The method for waterproofing below-grade structures according to claim 13, wherein said compound includes an expanding compound and/or a diffusing compound.

17. The method according to claim 16, wherein said expanding compound is selected from the group comprising:
a polyurethane resin;
a urea resin;
a silicone foam; or
mixtures thereof.

18. The method according to claim 16, wherein said diffusing compound is selected from the group comprising:
a silicate mixture;
a polyester resin;
an epoxy resin;
or mixtures thereof.

19. The method according to claim 13, wherein said injection step is performed at portions of said region of soil that have a lower resistivity than a first reference value.

20. The method according to claim 13, wherein said step of measuring the electrical resistivity of said at least one region of soil subjected to said injection step comprises a step of insertion, through respective openings for passing through a masonry and along a perimeter of said region of soil to be waterproofed, of a plurality of detection electrodes connected to a georesistivity meter.

21. The method according to claim 20, wherein said detection electrodes have a covering made of insulating material at least at the portion that is intended, during use, to arrange itself along the transverse extension of said masonry.

22. The method according to claim 13, wherein said at least one compound includes components and/or additives adapted to increase the resistivity of the respective compound.

23. The method according to claim 22, wherein said components and/or additives are adapted to increase the resistivity of the respective compound when it solidifies or gels.

24. The method according to claim 22, wherein said components and/or additives are selected from the group comprising:
acrylates;
acrylic copolymers;
or mixtures thereof.
